(19) United States
(12) Patent Application Publication
(10) Pub. No.: US 2019/0167598 A1
(43) Pub. Date: Jun. 6, 2019

(54) A NOVEL TREATMENT REGIMEN INVOLVING SAPROPTERIN DIHYDROCHLORIDE

(71) Applicant: DIPHARMA S.A., Chiasso (CH)

(72) Inventors: Gilles PAIN, Como (IT); Fabio CANNATA, San Fermo della Battaglia (IT); Bareket GELBHART, Riva San Vitale (CH)

(21) Appl. No.: 16/321,197

(22) PCT Filed: Jul. 27, 2017
(86) PCT No.: PCT/EP2017/068993

§ 371 Date: Jan. 28, 2019

Related U.S. Application Data
(60) Provisional application No. 62/368,341, filed on Jul. 29, 2016.

(51) Int. Cl. A61K 9/20; A61P 1/16; A61K 31/4965

(52) U.S. Cl. CPC A61K 9/2086; A61P 1/16; A61K 31/375; A61K 9/2027; A61K 31/4965; A61K 9/2018

(57) ABSTRACT

The invention relates to an improved tetrahydrobiopterin formulation for treating patients affected by hyperphenylalaninemia. More specifically, the formulation of the invention permits a prolonged residence of tetrahydrobiopterin in the plasma, and/or a higher concentration of sapropterin in the plasma, enabling notably the treatment of HPA patients that would usually not be classified as responders to tetrahydrobiopterin loading test, and therefore left without any pharmacological treatment opportunity, but only with a strictly controlled protein diet.

### FIELD OF THE INVENTION

The present invention relates to a novel tetrahydrobiopterin formulation for treating patients affected by hyperphenylalaninemia.

More specifically, the novel formulation encompasses tablet, capsule, pill, dragee, granule, sachet, powder, microparticulate system, solution, syrup, slurry, suspension, and the like, containing sapropterin and an antioxidant, the latter being such, or formulated in such a way to enable its extended-release in the gastro-intestinal tract.

### BACKGROUND OF THE INVENTION

Phenylketonuria (PKU) is an autosomal recessive hereditary metabolic disorder caused by genetic mutation(s) in a liver enzyme named phenylalanine hydroxylase (PAH), the enzyme that metabolizes phenylalanine (Phe) into tyrosine. Defect in said metabolism pathway inevitably leads to hyperphenylalaninemia (HPA) which in turn leads to severe neurological impairment. PKU is the cause of almost all (about 98%) cases of hyperphenylalaninemia (HPA) (Blau N., et al., Molec. Genet. Metab., 2009, 96, 158). A minority of cases also arises from impairment of PAH co-factor biosynthesis (i.e., tetrahydrobiopterin, thereafter referred as BH₄.2HCl or sapropterin) or from its regeneration.

PKU is a rare disorder, meaning that it affects less than 5 people over 10,000 in Europe, less than 200,000 in USA and less than 50,000 in Japan. An incidence of about 1 in 10,000 in individuals of European and Oriental Asian origin has been reported (Scriver C. R., Human Mutation, 2007, 28, 9, 831) with some populations more affected than others. For example, the incidence of PKU in Scottish population is estimated to 1:5300, in Italy 1:17000 and only 1:125000 in Japan (Scriver C. R., et al., The Metabolic and Molecular Bases of Inherited Disease; 8^{th} ed. New York: McGrawHill; 2001, 1667).

In patients affected by PAH deficiency while on a normal protein intake, HPA severity can be classified based on blood phenylalanine concentration in three main categories: classical PKU (phenylalanine>1200 μmol/l); moderate PKU (phenylalanine 600 to 1200 μmol/l); and mild PKU (phenylalanine<600 μmol/l but>360 μmol/l). 360 μmol/l is generally considered as the threshold above which damages occur and above which patients should be tested for potential sapropterin treatment (Vockley J., et al., Genet. Med., 2014, 16, 2, 188).

Some researchers classify the severity of the disease in a more detailed way considering also Phe tolerance as detailed in the table underneath (Scala I., et al., Orphanet J. Rare Dis., 2015, 10, 14).

| Phe level (μmol) | PKU severity | Phe tolerance (mg/day) |
|---|---|---|
| >1200 | classical | <350 |
| 900-1200 | moderate | 350-400 |
| 600-900 | mild | 400-600 |
| 360-600 | mild HPA-grey zone | >600 |

Sapropterin as an active ingredient has been used since late 70's by Dr. Schircks to treat patients affected by PKU on a name-patient basis (Schaub, J., et al., 1978, Arch. Dis. Child., 53, 8, 674). In 1980 Schircks Laboratories was set up by Dr. B. Schircks to produce tablets containing 10 mg and 50 mg sapropterin for treating patients on a compassionate use, named-patient basis worldwide. However, it was not until 1999 that sapropterin produced by Schircks Laboratories was provided with less than 0.5% of the much less active isomer 6S. At that time, the formulation consisted of tablets comprising a combination of more than 95% of the most active isomer 6R-BH₄.2HCl, less than 5% of the less active 6S-BH₄.2HCl and ascorbic acid in a 1:1 (w/w) ratio. Usual treatments consisted of administering between 5 to 20 mg/kg/day of sapropterin dihydrochloride.

In a long-term study, treatment with Schircks' sapropterin tablets (i.e., 20 mg/kg/day) increased phenylalanine tolerance in children with severe phenotype of phenylketonuria. Furthermore, their daily median phenylalanine tolerance increased from 18 mg/kg to 40 mg/kg (Hennermann J. B., et al., Mol. Genet. Metab., 2005, 86, S86). At that time, because of the short sapropterin elimination half-life time of about 4 h, it was suggested to patients with BH₄ deficiency and BH₄-responsive HPA/PKU, that BH₄ should have been administered in at least 2-3 daily doses in order to optimize the therapeutic response (Fiege B., et al., Molec. Genet. Metab., 2004, 81, 45).

Then, a formulation from Biomarin Pharmaceuticals Inc., different from the one developed by Dr. Schircks, was approved in USA in December 2007 and in Europe the year after (i.e., Kuvan®). This formulation contains around 4.6 mg of ascorbic acid for 100 mg of BH₄.2HCl. U.S. Pat. No. 7,566,462, filed in name of Biomarin Pharmaceuticals Inc., claims a stable tablet formulation comprising sapropterin and ascorbic acid, wherein the ratio of ascorbic acid/sapropterin ranges from about 1:5 to about 1:30.

EP1708690, filed in name of Biomarin Pharmaceuticals Inc., claims a once a day treatment regimen of sapropterin. European Kuvan® SmPC also recommends that the medicine should be taken together with a high calorie meal, since it is declared that high-calorie meal resulted in average, in 40-85% higher sapropterin maximum blood concentrations achieved 4 to 5 hours after administration as compared to fasting conditions.

However, if taking the medicine once a day instead of two to three times a day, could be seen as a more compliant treatment, it is however undoubtable that most PKU patients do not see any benefit from this treatment and are consequently obliged to follow an extremely strict diet as the only option to maintain a sustainable level of Phe. It has been reported lately, that in clinical studies, response to sapropterin was neither consistent nor predictable, and that some subjects in phase 2 and 3 trials demonstrated that the effect of sapropterin may not be sustained (Peters S., et al., Clinical Medicine Insights: Therapeutics, 2011, 3, 327). PKU is indeed a disease for which an urgent unmet medical need still exists, since most of the patients detected with PKU do not respond to the unique treatment approved and available today. Indeed, it is estimated that only around 20% of the patients can see their Phe level decreased by at least 30%, an arbitrary threshold recognized by the scientific community as the evidence of effective Phe reduction, upon regular administration of Kuvan® (Burton B. K., et al., J. Inherit. Metab. Dis., 2007, 30, 5, 700). Few reasons can explain why no real effective treatment has yet been identified. As anticipated, PKU results from genetic modification(s) of PAH enzyme. On May 23, 2016, the open-access PAHvdb database, reported 955 variants of the PAH gene, meanwhile the last update made on Mar. 6, 2017 reports 991 PAH variants (http://www.biopku.org/home/pah.asp). Unfortunately, no clear relationship between genotype and responsiveness to sapropterin supplementation has been recognized so far (Sarkissian C. N., et al, J. Inherit. Metab. Dis., 2012, 2, 5, 59). This is potentially due to the fact that since responsiveness is measured as percentage decrease of Phe level and that diet cannot be precisely controlled (patients are usually not hospitalized), any shift in Phe level could, at least partially, be due to unmonitored diet. Besides this parameter, it has also been demonstrated that the current treatment showed huge inter- and intra-patient pharmacokinetic variability (Blau N., et al., Mol. Genet. Metab., 2004, 82, 101; Fiege B., et al., Mol. Genet. Metab., 2004, 81, 45; Burton B. K., et al., J. Inherit. Metab. Dis., 2007, 30, 5, 700).

Patent application EP1964566 filed in name of Asubio Pharma Co., Ltd., disclosed an improved bio-absorbability of sapropterin through enhanced intestinal drug absorption by means of formulations comprising organic carboxylic acids having more than one carboxyl group, such as tartaric acid, citric acid and malic acid. However, it was clear from the examples that even with an amount of citric acid 3.2 times higher than sapropterin (w/w), blood levels of BH₄ were inferior to that observed from the control group.

A further known shadow of sapropterin resides in its chemical instability. Indeed, sapropterin is highly hygroscopic and is also readily oxidized into dihydrobiopterin in neutral or basic conditions. The CHMP assessment report of Kuvan® issued by EMA mentions that ascorbic acid present in the formulation has been shown to prevent the auto-oxidation of BH₄ to BH₂. However, it has also been suggested that, rather than preventing auto-oxidation of BH₄, ascorbic acid exerts a direct reducing effect on the oxidation product of BH₄, presumably on qBH₂ (Tóth M., et al., Molec. Hum. Reprod., 2002, 8, 3, 271). Ascorbic acid is better known as vitamin C.

The clinical trial website of the U.S. National Institutes of Health reports about 69 clinical trials completed or on-going on PKU, clearly demonstrating that new methods of treatment are still required.

Consequently, there is an urgent medical need to provide effective treatment, notably to patients who actually do not respond to sapropterin supplementation. Furthermore, there is also the need to find a method of treatment that could minimize or even abolish the inter- and intra-individual patient pharmacokinetic variability observed in numbers of patient under the currently unique approved HPA treatment.

### DESCRIPTION OF THE INVENTION

It has now been surprisingly found a new pharmaceutical composition kit for use in a treatment regimen comprising two main pharmaceutically active ingredients, sapropterin and an antioxidant, the latter being such that its concentration in the body will remain at a therapeutically effective level for a period of time up to about 24 hours, preferably up to about 6 to 12 hours or up to about 6 to 10 hours, or up to about 8 to 10 hours.

Such treatment regimen can also benefit to HPA patient population so far left without any medical option.

For the purpose of the present invention, the pharmaceutical composition kit is made of two different pharmaceutical formulations referred later to type a) and type b); each one containing one main pharmaceutically active ingredient defined as sapropterin and as antioxidant respectively. It will become clear to the skilled person that the two different pharmaceutical formulations referred to type a) and type b) can be in two separate dosage forms or in a unique dosage form. In the latter case, the pharmaceutical composition will still be referred as to a pharmaceutical composition kit.

For the purpose of the present invention, depending on the context, the skilled person will easily understand that the expression “pharmaceutical formulation” can be synonymous to “pharmaceutical composition kit” or “pharmaceutical composition”.

For the purpose of the present invention, the terms “sapropterin”, “sapropterin dihydrochloride”, “BH₄”, and “BH₄.2HCl” are intended to be synonymous, and unless expressly mentioned otherwise, all refer to the chemical compound (6R)-2-amino-6-[(1R,2S)-1,2-dihydroxypropyl]-5,6,7,8-tetrahydropteridin-4(1H)-one dihydrochloride.

For the purpose of the present invention, the expression “stabilizing agent” is intended to notably encompass any compound that can stabilize tetrahydrobiopterin, and include, but are not limited to, antioxidants, chelating agents, moisture retaining agents, disaccharides or higher polyols, hydrophobic agents and the like, cyclodextrins, or any combinations thereof.

For the purpose of the present invention, the term “antioxidant” is intended to notably encompass any of the following compounds: citric acid, tartaric acid, vitamin C, plant polyphenols, anthocyanins, flavonoids, isoflavonoids, glutathione and the like. The antioxidant agents described herein are not intended to constitute an exhaustive list, but are provided merely as exemplary antioxidant agents that may be used in the present invention.

For the purpose of the present invention, the term “antioxidant” when employed without any specific reference to pharmaceutical formulation of type a) or of type b), refers to the antioxidant of the pharmaceutical formulation type b).

For the purpose of the present invention, the expression “Phe baseline level” refers to phenylalanine level measured from a blood sample taken from an untreated patient in a fasting state.

For the purpose of the present invention, the expression “untreated patient” refers to a patient suffering from HPA who has never received BH₄, or who has not received BH₄ for at least a week before blood test is performed.

For the purpose of the present invention, the expression “extended-release” means controlled-release, sustained-release, delayed-release or long-acting formulation, and should refer to a period of time up to about 24 hours, preferably up to about 6 to 12 hours or up to about 6 to 10 hours, or up to about 8 to 10 hours.

For the purpose of the present invention, the expression “fast release” is synonymous of immediate release.

For the purpose of the present invention, the term “fasting” shall be understood to refer to a period of from about 1 hour before or about 2 hours after the consumption of food.

For the purpose of the present invention, the term “about” as used herein, when referring to a numerical value or range, refers to a degree of variability in the value or range, for example, within 10%, or within 5% of the stated value or of the stated limits of a range.

For the purpose of the present invention, the terms “co-administering” and “co-administration” refer either to simultaneous or sequential administration.

In case of simultaneous administration, the pharmaceutical composition kit contains two main pharmaceutical compositions (type a) and type b)) with potentially different release properties and is intended to be administered once a day, preferably in a fasting state.

The pharmaceutical composition type a) mainly contains sapropterin as the principal pharmaceutically active ingredient together with a stabilizing agent, and is formulated in such a way that sapropterin is immediately released once administered; meanwhile the second pharmaceutical composition (type b)) mainly contains an antioxidant as the principal pharmaceutically active ingredient and is formulated in such a way that the antioxidant concentration remains at a therapeutically effective level for a period of time up to about 24 hours, preferably up to about 6 to 12 hours or up to about 6 to 10 hours, or up to about 8 to 10 hours.

In case the antioxidant of pharmaceutical composition type b) has a short half-life, it can be formulated as an extended-release formulation to be released over an extended period of time up to about 24 hours, preferably up to about 6 to 12 hours or up to about 6 to 10 hours, or up to about 8 to 10 hours.

In one preferred embodiment of the present invention, the stabilizing agent is present in an amount up to 5% w/w that of sapropterin, preferably in an amount up to 2.5%.

In a more preferred embodiment of the present invention, the stabilizing agent is an antioxidant which can be identical or different to the antioxidant present in the pharmaceutical composition type b).

In an even more preferred embodiment of the present invention, the antioxidant of the pharmaceutical composition type b) is chosen from citric acid, tartaric acid, vitamin C, plant polyphenols, anthocyanins, flavonoids, isoflavonoids and glutathione; vitamin C being preferred.

In one more preferred embodiment of the present invention within the context of simultaneous co-administration, the above-mentioned pharmaceutical compositions type a) and type b) can be in the form of tablet, capsule, pill, dragee, granule, sachet, powder, microparticulate system, solution, syrup, slurry, suspension, and the like.

In one even more preferred embodiment of the present invention within the context of simultaneous co-administration, the above-mentioned pharmaceutical compositions type a) and type b) are granules, sachet, powder, or microparticulate system that could be suspended in a liquid before administration, such as water, milk or fruit juice.

In a still even more preferred embodiment of the present invention within the context of simultaneous co-administration, the liquid for suspending the granules, sachet, powder, or microparticulate system is water.

In a second more preferred embodiment of the present invention within the context of simultaneous co-administration, the above-mentioned pharmaceutical compositions type a) and type b) are tablets for oral absorption with specific, and potentially different, coatings to enable the release of the two pharmaceutically active ingredients in such a way that the antioxidant concentration contained in the pharmaceutical composition type b) remains at a therapeutically effective level for a period of time up to about 24 hours, preferably up to about 6 to 12 hours or up to about 6 to 10 hours, or up to about 8 to 10 hours.

In a more preferred embodiment of the present invention within the context of simultaneous co-administration, the stabilizing agent contained in the pharmaceutical composition type a) is an antioxidant, preferably ascorbic acid; meanwhile the antioxidant contained in the pharmaceutical composition type b) of the tablets is ascorbic acid, the latter being formulated as a delayed-release formulation.

In case of sequential co-administration, the principal two pharmaceutically active ingredients, sapropterin and antioxidant, can be formulated either with similar immediate release properties (first case scenario), or with different release properties (second case scenario).

In the first case scenario, sapropterin is possibly administered only once a day, meanwhile the antioxidant, in case of a short half-life, is administered firstly within 30 minutes before or after sapropterin, before being preferred, and then at regular intervals between two and six times a day, preferably between 2 and 4 times a day.

In this first case scenario, the antioxidant is preferably citric acid, tartaric acid, or a vitamin, and more preferably is ascorbic acid.

In another embodiment of this first case scenario, sapropterin is possibly administered only once a day as an immediate-release formulation, as well as the antioxidant. In a preferred embodiment, the antioxidant is administered within 30 minutes before or after sapropterin, before being preferred. In a more preferred embodiment, the antioxidant has a half-life of at least 6 hours, preferably of at least 8 hours and even more preferably of at least 12 hours.

In the second case scenario, sapropterin is possibly administered only once a day as an immediate-release formulation, and the antioxidant is possibly administered only once a day in such a way that the antioxidant concentration remains at a therapeutically effective level for a period of time up to about 24 hours, preferably up to about 6 to 12 hours or up to about 6 to 10 hours, or up to about 8 to 10 hours.

In a preferred embodiment of this second case scenario, the antioxidant is a vitamin, and is formulated as a delayed-release formulation and is administered within 30 minutes before or after administration of sapropterin, 30 minutes before being preferred.

In a more preferred embodiment of this second case scenario, the antioxidant is ascorbic acid.

In an even more preferred embodiment of this second case scenario, the administration of the pharmaceutical formulation occurs in a fasting state.

In one other preferred embodiment of the present invention within the context of sequential co-administration, the above-mentioned sapropterin and antioxidant can be formulated as tablets, capsules, pills, dragees, granules, sachets, powders, microparticulate systems, solutions, syrups, slurries, suspensions, and the like; being granules, sachets, powders, capsules and tablets preferred.

In a more preferred embodiment of the present invention within the context of sequential co-administration, the two pharmaceutically active ingredients are formulated as sachets or tablets.

Another embodiment of the present invention consists of a pharmaceutical composition kit comprising:
a) a fast-release formulation containing sapropterin dihydrochloride and a stabilizing agent in an amount up to about 5% w/w that of sapropterin, more preferably up to about 2.5% w/w, and
b) a formulation containing an amount of antioxidant from about 40% to about 100% w/w that of the sapropterin contained in the fast-release formulation type a),
wherein the pharmaceutical formulation kit can be in a unique dosage form or in two separate dosage forms.

A more specific embodiment of the present invention consists of the above-mentioned pharmaceutical composition kit wherein formulation a) containing sapropterin dihydrochloride is to be administered in a fasting state at a daily dose of 5 mg/kg to 20 mg/kg, and wherein formulation b) containing an amount of antioxidant from about 40% to about 100% w/w that of the sapropterin contained in the fast-release formulation a) is to be administered at a daily dose of 2 mg/kg to 20 mg/kg.

In a preferred embodiment of the present invention, the stabilizing agent of formulation a) and the antioxidant of formulation b) are ascorbic acid.

In a further more specific embodiment of the present invention within the context of simultaneous co-administration, the pharmaceutical composition kit is such that formulations a) and b) are in a unique dosage form and formulation b) is formulated in such a way that the antioxidant concentration remains at a therapeutically effective level for period of time up to about 24 hours, preferably up to about 6 to 12 hours or up to about 6 to 10 hours, or up to about 8 to 10 hours.

In a preferred embodiment of the present invention within the context of simultaneous co-administration, the antioxidant of formulation b) is ascorbic acid.

In an even further more specific embodiment of the present invention within the context of simultaneous co-administration, the pharmaceutical composition kit is in the form of tablets, granules, capsules, pills, dragees, sachets, powder or microparticulate systems.

In a further preferred embodiment of the present invention within the context of simultaneous co-administration, the pharmaceutical composition kit is in the form of tablets.

In a further preferred embodiment of the present invention within the context of simultaneous co-administration, the pharmaceutical composition kit is in the form of granules, sachets, powder or microparticulate systems, wherein said granules, sachets, powder or microparticulate systems can be suspended in a liquid, such as water, milk or fruit juice for oral administration.

Another embodiment of the present invention, consists of a pharmaceutical composition kit for sequential co-administration wherein formulations a) and b) are in two different dosage forms and wherein formulation b) is formulated in such a way to enable immediate-release of the antioxidant, and wherein formulation b) is to be administered from 2 to 6 times per day at regular intervals of about 2 hours in an amount of between 0.33 mg/kg to 10 mg/kg each time, wherein the first supplementation of antioxidant occurs in a fasting state within 30 minutes, before or after intake of sapropterin.

In a preferred embodiment of the present invention within the context of sequential co-administration, formulation a) is administered in a fasting state. In a more preferred embodiment of the present invention within the context of sequential co-administration, immediate-release formulation b) is administered 4 times per day in an amount of between 0.5 mg/kg to 5 mg/kg each time.

In another preferred embodiment of the present invention within the context of sequential co-administration, immediate release formulation b) is administered the first time within 30 minutes before intake of sapropterin.

In another further preferred embodiment of the present invention within the context of sequential co-administration, immediate release formulation b) contains ascorbic acid as the main active ingredient.

Another preferred embodiment of the present invention relates to a novel treatment regimen aimed at targeting a HPA patient population also including patients declared as non-responders to sapropterin treatment when checked through a 24-hour BH₄ loading test as disclosed for example in Bernegger C., et al., Mol. Genet. Metab., 2002, 77, 304; and/or including patients declared as non-responders to sapropterin treatment when checked through a 48-hour loading test as disclosed for example in Scala I., et al., Orphanet J. Rare Dis., 2015 February, 10:14; and/or including patients declared as non-responders to sapropterin treatment when checked through over a one month period as recommended in the European summary of product characteristics of Kuvan®.

In a particularly more preferred embodiment of the present invention, the HPA patient population targeted by the present invention mainly consists of patients declared as non-responders to sapropterin treatment when checked through a 48-hour BH₄ loading test as disclosed above.

In another embodiment of the present invention, the targeted HPA patient population consists of patients having a Phe baseline level above 1200 μmol/l, or between 600 to 1200 μmol/l, and more preferably patients having a Phe baseline level between 600 to 1200 μmol/l or above 1200 μmol/l who do not see a decrease of Phe level of at least 30% after a 24-hour or 48-hour BH₄ loading test or after at least a week of currently approved sapropterin treatment.

In another more specific embodiment of the present invention, the HPA patient population targeted is affected by PKU.

### Treatment Regimen A

A preferred embodiment of the present invention, relates to a treatment regimen of a patient affected by HPA, said treatment regimen comprising:
a) administering once a day in a fasting state, sapropterin immediate release formulation containing sapropterin dihydrochloride as the main pharmaceutically active ingredient, at a dose of between 5 mg/kg to 20 mg/kg; and
b) co-administering an antioxidant immediate release formulation from 2 to 6 times per day at regular intervals of about 2 hours in an amount of between 0.33 mg/kg to 10 mg/kg each time, wherein the first supplementation of the antioxidant occurs within 30 minutes, before or after intake of sapropterin.

Another preferred embodiment of the present invention, relates to treatment regimen A herein-above, wherein the total daily amount of the antioxidant of step b) can vary between 2 mg/kg to 20 mg/kg.

Another further preferred embodiment of the present invention, relates to treatment regimen A herein-above, wherein co-administration of the antioxidant of step b) occurs 4 times per day.

Another even further preferred embodiment of the present invention, relates to treatment regimen A herein-above, wherein the first co-administration of the antioxidant of step b) occurs before intake of sapropterin.

Another even further more preferred embodiment of the present invention, relates to treatment regimen A herein-above, wherein the first co-administration of the antioxidant of step b) occurs in the morning, before intake of sapropterin.

Another most preferred embodiment of the present invention, relates to treatment regimen A herein-above, wherein the antioxidant of step b) is vitamin C.

Another most preferred embodiment of the present invention, relates to treatment regimen A herein-above, wherein sapropterin immediate release formulation of step a) further contain up to 5% w/w with respect to that of sapropterin of a stabilizing agent, the latter being preferably ascorbic acid.

### Treatment Regimen B

Another preferred embodiment of the present invention, relates to a treatment regimen of a patient affected by HPA, said treatment regimen comprising administering once a day, in a fasting state a pharmaceutical formulation in the form of a liquid suspension obtained by suspending in a liquid, such as water, milk or fruit juice, water being preferred, a powder, sachet, microparticulate system, or granules, wherein part of the powder, sachet, microparticulate system or granules mainly contains sapropterin as the principal pharmaceutically active ingredient for immediate release at a daily dose of between 5 mg/kg to 20 mg/kg, and wherein the other part of the powder, sachet, microparticulate system or granules mainly contains an antioxidant as the principal pharmaceutically active ingredient, for delayed-release at a daily dose of between 2 mg/kg to 20 mg/kg.

Another preferred embodiment of the present invention, relates to treatment regimen B herein-above, wherein the pharmaceutical formulation is taken in the morning.

Another more preferred embodiment of the present invention, relates to treatment regimen B herein-above, wherein the antioxidant formulated as a delayed-release formulation is vitamin C.

Another more preferred embodiment of the present invention, relates to treatment regimen B herein-above, wherein the formulation containing sapropterin as the main pharmaceutically active ingredient further contains a stabilizing agent in an amount up to 5% w/w that of sapropterin, the former being preferably ascorbic acid.

### Treatment Regimen C

Another preferred embodiment of the present invention, relates to a treatment regimen of a patient affected by HPA, said treatment regimen comprising administering once a day, in a fasting state a pharmaceutical formulation in the form of a tablet, wherein the tablet comprises:
a) a fast-release composition containing sapropterin dihydrochloride as the main pharmaceutically active ingredient in an amount of 100 mg, and
b) a sustained-release composition containing an amount of antioxidant of at least 40 mg±10% and potentially up to 100 mg±10%,
wherein, the treatment regimen enables the daily delivery of between 5-20 mg/kg of sapropterin dihydrochloride and of between 2 mg/kg to 20 mg/kg of antioxidant.

Another further preferred embodiment of the present invention, relates to treatment regimen C herein-above, wherein the fast-release composition (type a)) further contains up to 5% w/w with respect to that of sapropterin of a stabilizing agent, the latter being preferably ascorbic acid.

Another even further preferred embodiment of the present invention, relates to treatment regimen C herein-above, wherein the antioxidant contained in the sustained-release composition is vitamin C.

Another even further more preferred embodiment of the present invention, relates to treatment regimen C herein-above, wherein the pharmaceutical formulation is taken in the morning.

Another preferred embodiment of the present invention contemplates alleviating the phenylalanine-restricted diet of mild, moderate and/or classic PKU patients, through any one of treatment regimen A to C herein-above mentioned, affording the patients the opportunity to assume more protein intake daily with respect to the dose usually recommended meanwhile being under currently approved sapropterin treatment regimen.

A further more preferred embodiment of the present invention contemplates the possibility to reduce sapropterin dosage in order to obtain a similar decrease of Phe level with respect to the one that would be obtained through currently approved sapropterin treatment regimen. Such a lower dosage may benefit to patient population having evidenced side-effects upon exposure to currently approved sapropterin regimen and will concur to substantially reduce the overall cost of the treatment.

Without wanting to be bound by any theory, it is believed that the treatment regimen object of the present invention derives its surprising benefits from a prolonged residence of sapropterin in the plasma, and/or a higher concentration of sapropterin in the plasma, compared to what that could be obtained through a similar treatment regimen making use of a similar pharmaceutical formulation but not enabling a prolonged presence of an antioxidant.

A specific embodiment of the present invention resides in a formulation comprising BH₄ and further containing an extended-release composition containing ascorbic acid. The formulation can be in tablet form, granules, capsule, pill, dragee, sachet, in microparticulate system, solution, syrup, slurry, suspension, and the like, for ingestion by the patient.

Tablet form, sachet and suspension are preferred formulations.

### Tablet Form for Simultaneous Co-Administration

A specific embodiment of the present invention resides in a tablet formulation, comprising:
a) a fast-release external formulation containing sapropterin dihydrochloride as the main pharmaceutically active ingredient in an amount of 100 mg, and
b) a sustained-release internal formulation containing an amount of antioxidant of at least 40 mg±10% w/w with respect to that of sapropterin and potentially up to 100 mg±10% w/w with respect to that of sapropterin.

In a more specific embodiment of the present invention, the fast release external formulation of the tablet herein-above mentioned further contains up to about 5% w/w of ascorbic acid with respect to that of sapropterin dihydrochloride, and preferably up to about 2.5% w/w.

In a preferred embodiment, the pharmaceutical composition enabling extended antioxidant plasma concentration, contains an amount of antioxidant in the sustained-release formulation of about 40% (w/w), or of about 50% (w/w), or of about 60% (w/w), or of about 70% (w/w), or of about 80% (w/w), or of about 90% (w/w), or of about 100% (w/w) that of sapropterin contained in the fast-release formulation.

In a more preferred embodiment, the pharmaceutical composition enabling extended antioxidant plasma concentration, contains an amount of antioxidant in the sustained-release formulation of about 40% (w/w), or of about 50% (w/w), or of about 60% (w/w) that of sapropterin contained in the fast-release formulation.

In an even more preferred embodiment, the pharmaceutical composition also enables a higher concentration of sapropterin in the plasma and/or an extended sapropterin plasma concentration with respect to a formulation not containing a sustained-release formulation of antioxidant.

In a most preferred embodiment, the antioxidant contained in the sustained-release formulation is vitamin C.

The sustained-release formulation can be made of well-known ingredients such as for example, sustained-release polymers derived from cellulose; polysaccharide family; gum family; peptide derivatives such as collagen or gelatin; or acrylic acid derivatives.

### Suspension Form for Simultaneous Co-Administration

Another preferred embodiment of the present invention provides a pharmaceutical composition kit in powder, microparticulate system or granules form, preferably presented in sachet, to be suspended in a liquid before administration, comprising two types of granules, microparticulate systems or powders (i.e., type a) and type b)) as follows:
type a) granules, microparticulate systems or powders containing sapropterin as the main pharmaceutically active ingredient, wherein said granules, microparticulate systems or powders embodying sapropterin enable an immediate release of the latter; and
type b) granules, microparticulate systems or powders containing an antioxidant as the main pharmaceutically active ingredient and no sapropterin, wherein said granules, microparticulate systems or powders embodying the antioxidant enable an antioxidant concentration that remains at a therapeutically effective level for a period of time up to about 24 hours, preferably up to about 6 to 12 hours or up to about 6 to 10 hours, or up to about 8 to 10 hours.

In a more preferred embodiment, the antioxidant contained in the granules, microparticulate systems or powders of type b) is vitamin C and is formulated as a sustained-release formulation.

In another more preferred embodiment, the granules, microparticulate systems or powders of type b) are coated through well-known methods such as for example pan coating, spray drying, solvent evaporation, fluidised bed technology, solvent-less dry-polymer coating, or coaservation-phase separation. In an even more preferred embodiment, the pharmaceutical composition kit above-mentioned is dispersible in water, milk or fruit juice before administration.

### Suspension Form for Sequential Co-Administration

Another preferred embodiment of the present invention provides a pharmaceutical composition in powders, microparticulate systems or granules form, preferably presented in two separate packaging, comprising two types of granules, microparticulate systems or powders (i.e., type c) and type d)) as follows:
type c) in a first packaging, the granules, microparticulate systems or powder contain sapropterin as the main pharmaceutically active ingredient, and said granules, microparticulate systems or powder embodying sapropterin enable an immediate release of the latter; and
type d) in a second packaging, the granules, microparticulate systems or powder contain an antioxidant as the main pharmaceutically active ingredient and no sapropterin, and said granules, microparticulate systems or powder embodying the antioxidant enable an immediate release of the latter,
wherein the granules, microparticulate system or powder of type d) are to be administered to a patient in need thereof, from 2 to 6 times per day at intervals of about 2 hours in an amount from about 0.33 mg/kg to about 10 mg/kg of ascorbic acid each time, wherein the first administration occurs within about 30 minutes, before or after the administration of the granules, microparticulate system or powder embodying sapropterin of the first packaging.

### EXAMPLES

### Example 1

### Sapropterin Tablet Formulation Content

| Ingredients | Quantity (mg) |
|---|---|
| Sapropterin dihydrochloride | 100.00 |
| Ascorbic acid | 2.30 |
| Sodium stearyl fumarate | 9.00 |
| Copovidone | 30.00 |
| Mannitol | 119.65 |
| Crospovidone | 36.00 |
| Riboflavin fine powder | 0.05 |
| Colloidal silicon dioxide | 3.00 |

Tablets having a total weight of 300.00 mg each were formulated following standard procedures.

### Example 2

Two groups of 10 patients affected by PKU, with a mean body weight of 61 kg±10%, each under a controlled diet are given sapropterin as formulated according to example 1 at a dose of 10 mg/kg/day, 1 hour before breakfast without any additional supplement of ascorbic acid (Group I) or with additional supplement of ascorbic acid (Group II) according to a randomization scheme. Ascorbic acid supplementation is effected 4 times per day (60 mg×4 times) at 2 hour intervals, with the first supplementation within 30 minutes (before or after) of intake of sapropterin. Each group receives such treatment for 30 days. At the end of these first 30 days (Period 1), a washout period of 1 day is attended and the study groups are then crossed-over to enter into Period 2 treatment. All patients continue to receive the same daily dose of sapropterin during the washout period. Consequently, patients of Group I receives a dose of sapropterin formulated according to example 1, 10 mg/kg/day for 30 days, 1 hour before breakfast with additional supplement of ascorbic acid given 4 times per day (60 mg×4 times) at 2 hour intervals, with the first supplementation within 30 minutes (before or after) of intake of sapropterin. Patients of Group II receives a dose of sapropterin formulated according to example 1, 10 mg/kg/day for 30 days, 1 hour before breakfast without any additional supplement of ascorbic acid.

Phe plasmatic levels of each patient is monitored in both periods at day 0 (prior to first administration of sapropterin and/or ascorbic acid), then at day 8 and finally at day 30.

### Example 3

The aim of study of example 2 is to show that:
a) patients from Group II/Period 1 and patients from Group I/Period 2 demonstrate a more significant decrease of their Phe levels than patients from Group I/Period 1 or from Group II/Period 2, both at day 8 and at day 30;
b) meanwhile only less than 20% of patients from Group I/Period 1 having a Phe level baseline between 600 to 1200 μmol/l see their Phe levels decreasing by at least 30% at day 8 and/or at day 30; when following the second period (Group I/Period 2) the percentage of patients showing a decrease of Phe level greater or equal to 30% is approximatively between 40% and 70% depending on the initial disease severity;
c) the observations mentioned at item b) are corroborated with Phe levels of patients from Group II who demonstrate a much higher response in Period 1 than in Period 2, response comparable to that seen from patients belonging to Group I/Period 2;
d) when looking at sapropterin PK, patients from Group II/Period 1 and patients from Group I/Period 2 demonstrate Cmax and AUC values almost double with respect to patients from Group I/Period 1 and/or from patients from Group II/Period 2;
e) surprisingly, the effect described at item d) is reduced when the experiment of example 2 is done under fed conditions.

From the data described in example 2, a formulation, preferably in tablet, capsule, pill, dragee, granule, sachet, powder, or microparticulate system, comprising the ingredients as per example 1, and further containing an extended-release formulation containing from about 40 mg to about 100 mg of ascorbic acid appears suitable for use in the methods of treatment described herein.

### Example 4

Two groups of 15 patients affected by PKU, each under a controlled diet are given sapropterin as formulated according to example 1 at a dose of 20 mg/kg/day, 1 hour before breakfast without any additional supplement of ascorbic acid (Group I) or with additional supplement of ascorbic acid (Group II) according to a randomization scheme. Ascorbic acid supplementation is effected 4 times per day (total daily dose of 10 mg/kg) at 2 hour intervals, with the first supplementation within 30 minutes before intake of sapropterin. Each group receives such treatment for 8 days. At the end of these first 8 days (Period 1), a washout period of a full week is observed and the study groups are then crossed-over to enter into Period 2 treatment. All patients continue to receive the same daily dose of sapropterin during the washout period. Consequently, patients of Group I receives a dose of sapropterin formulated according to example 1, 20 mg/kg/day for 8 days, 1 hour before breakfast with additional supplement of ascorbic acid given 4 times per day (total daily dose of 10 mg/kg) at 2 hour intervals, with the first supplementation within 30 minutes before intake of sapropterin. Patients of Group II receives a dose of sapropterin formulated according to example 1, 20 mg/kg/day for 8 days, 1 hour before breakfast without any additional supplement of ascorbic acid.

Phenylalanine and tyrosine plasmatic levels of each patient is monitored in both periods as follows:
days 0 (day prior to first administration of sapropterin and/or ascorbic acid),
day 1 (blood samples at 4 h, 8 h, 16 h and 24 h after first administration of sapropterin),
day 2, day 4, day 6 and day 8 (single blood sampling).

### Example 5

The aim of study of example 4 is to show that:
a) patients from Group II/Period 1 and patients from Group I/Period 2 demonstrate a more significant decrease of their Phe levels than patients from Group I/Period 1 or from Group II/Period 2, at day 1. Such observation can be confirmed at day 8;
b) meanwhile only less than 20% of patients from Group I/Period 1 having a Phe level baseline above 1200 μmol/l see their Phe levels decreasing by at least 30% at day 8; when following the second period (Group I/Period 2) the percentage of patients showing a decrease of Phe level greater or equal to 30% is approximatively between 40% and 70% depending on the initial disease severity;
c) the observations mentioned at item b) are corroborated with Phe levels of patients from Group II who demonstrate a much higher response in Period 1 than in Period 2, response comparable to that seen from patients belonging to Group I/Period 2.

### Example 6

Sapropterin and ascorbic acid can be formulated as a bilayer tablet comprising a controlled release first layer containing up to about 100 mg of ascorbic acid, at least one release modifier capable of providing controlled release of the ascorbic acid for at least 6 hours after ingestion, a lubricant, a filler, etc. The second layer includes 100 mg of sapropterin, about 2.3 mg of ascorbic acid as well as commonly found tablet excipients, i.e. a filler, a disintegrant, a lubricant, or the like.

1.-13. (canceled)

14. A method of treating hyperphenylalaninemia, comprising administering daily:
a) once only, a first fast-release formulation containing from 5 mg/kg to 20 mg/kg of sapropterin and a stabilizing agent, the stabilizing agent being present in an amount of up to about 5% w/w of the sapropterin; and
b) a second formulation containing from 2 mg/kg to 20 mg/kg of an antioxidant; to a patient in need thereof.

15. The method of claim 14, wherein the stabilizing agent of the first fast-release formulation is selected from the group consisting of antioxidants, chelating agents, moisture retaining agents, disaccharides or higher polyols, hydrophobic agents and the like, cyclodextrins, and combinations thereof.

16. The method of claim 14, wherein the antioxidant of the second formulation is selected from the group consisting of: citric acid, tartaric acid, vitamin C, plant polyphenols, anthocyanins, flavonoids, isoflavonoids, glutathione and mixtures thereof.

17. The method of claim 16, wherein the second formulation is administered from 2 to 6 times per day in an amount of between 0.33 mg/kg to 10 mg/kg per dose, with the first dose being administered within 30 minutes before or after the administration of the first fast-release formulation.

18. The method of claim 17 wherein the second dose of the second formulation is administered after 2 hours from the first dose of the second formulation, and then any subsequent doses, if any, at regular intervals.

19. The method of claim 17, wherein the dose of the first fast-release formulation and the first dose of the second formulation are administered in a fasting state.

20. The method of claim 17, wherein the antioxidant of the second formulation is ascorbic acid.

21. The method of claim 16, wherein the first fast-release formulation and the second formulation are administered simultaneously.

22. The method of claim 14, wherein the patient has phenylketonuria (PKU).

23. The method of claim 16, wherein the second formulation is an extended release formulation, releasing the antioxidant for up to 24 hours after administration.

24. The method of claim 23, wherein the second formulation releases the antioxidant from 6 to 12 hours after administration.

25. The method of claim 23, wherein the first fast-release formulation and the second formulation are administered simultaneously.

26. The method of claim 25, wherein the first fast-release formulation and the second formulation are administered in a single dosage form.

27. The method of claim 23, wherein the antioxidant of the second formulation is ascorbic acid.

28. The method of claim 26, wherein the first fast-release formulation and the second formulation are formulated as a tablet, capsule, pill, dragee, powder, microparticulate system or in granule form.

29. The method of claim 26, wherein single dosage form is administered in a fasting state.

30. The method of claim 23, wherein the first fast-release formulation and the second formulation are administered sequentially, the second formulation being administered within 30 minutes before or after the administration of the first fast-release formulation.

31. The method of claim 30, wherein the second formulation is administered within 30 minutes before the administration of the first fast-release formulation.
